(19) United States
(12) Patent Application Publication
(10) Pub. No.: US 2021/0355169 A1
(43) Pub. Date: Nov. 18, 2021

(54) RECOMBINANT RSV LIVE VACCINE STRAIN AND PRODUCTION METHOD THEREFOR

(71) Applicant: SK BIOSCIENCE CO., LTD., Seongnam-si, Gyeonggi-do (KR)

(72) Inventors: Ki-weon SEO, Seongnam-si, Gyeonggi-do (KR); Eun-som KIM, Seongnam-si, Gyeonggi-do (KR); Teawoo KWON, Seongnam-si, Gyeonggi-do (KR); Seung-hye HONG, Seongnam-si, Gyeonggi-do (KR); Hun KIM, Seongnam-si, Gyeonggi-do (KR); Su Jeen LEE, Seongnam-si, Gyeonggi-do (KR)

(21) Appl. No.: 17/283,658

(22) PCT Filed: Oct. 14, 2019
(86) PCT No.: PCT/KR2019/013447

§ 371 Date: Apr. 8, 2021

(30) Foreign Application Priority Data
Oct. 12, 2018 (KR) 10-2018-0122162

(51) Int. Cl. C07K 14/005; C12N 7/00; C12N 15/86; A61K 39/155; A61K 39/165; A61K 39/17; A61K 39/295; A61P 31/14

(52) U.S. Cl. CPC C07K 14/005; A61K 2039/5254; C12N 15/86; A61K 39/155; A61K 39/165; A61K 39/17; A61K 39/295; A61P 31/14; C12N 2760/18522; C12N 2760/18521; C12N 2760/18422; C12N 2760/18122; C12N 2760/18562; C12N 2760/18551; C12N 2760/18543; C12N 7/00

(57) ABSTRACT

The present invention provides a gene recombinant respiratory syncytial virus (RSV) in which genes encoding the envelope proteins of an RSV are rearranged, wherein in the RSV, a gene encoding the fusion protein (F protein) derived from a heterologous virus belonging to the family Paramyxoviridae or the family Pneumoviridae is inserted between the genes respectively encoding the glycoprotein (G protein) and the F protein of the RSV, or the gene encoding the F protein of the RSV is substituted with a gene encoding the F protein of a heterologous virus belonging to the family Paramyxoviridae or the family Pneumoviridae. The recombinant RSV of the present invention can be used as an RSV vaccine strain, and can be used as a vaccine due to having excellent stability and safety.

### TECHNICAL FIELD

The present application claims priority to Korean Patent Application No. 10-2018-0122162 filed on Oct. 12, 2018 in the Republic of Korea, the disclosures of which are incorporated herein by reference. The present disclosure relates to an RSV live vaccine strain and a method for producing the same. More specifically, it relates to a recombinant RSV live vaccine and a method for preparing the same.

### BACKGROUND ART

Respiratory syncytial virus (RSV) is a very common, contagious virus that causes respiratory diseases. Particularly, it is the main cause of death of infants due to severe respiratory tract infections. Although infants are at highest risk for infection, it is also known to cause fatal respiratory diseases in immunocompromised patients and the elderly by causing infections of the respiratory tract. While it is the second cause of respiratory diseases next to influenza, it is known that deaths caused by RSV are about 1.3-2.5 times more than influenza in infants. According to the WHO's report in 2002, 64 million people are infected with RSV every year, and 160,000 of them die.

For defense against RSV, a vaccine using inactivated virus (inactivated vaccine) was developed first. However, the use of the inactivated vaccine has become impossible due to a severe side effect (ERD; enhanced respiratory disease). Therefore, researches have made efforts to develop a vaccine with excellent ability of inducing neutralizing antibodies without causing ERD.

Meanwhile, although a live vaccine is advantageous in that it has excellent ability of inducing neutralizing antibodies without causing ERD, vaccine development is difficult when considering stability and safety issues of the virus because RSV is very unstable (metastable).

RSV belongs to the subfamily Orthopneumoviridae of the family Pneumoviridae, order Mononegavirales. RSV is a medium-sized virus of 120-200 nm. It has a surface structure consisting of an envelope and three transmembrane proteins (F and G glycoproteins and SH protein), and has a linear, negative-sense, single-stranded RNA genome of about 15,000 nucleotides in length. RSV is divided into serotypes A and B based on the G protein. The genome of RSV encodes 11 proteins, and is composed of a portion encoding structural proteins and a portion encoding non-structural proteins. The non-structural proteins, nonstructural (NS) 1 and nonstructural (NS) 2, act on the immune escape mechanism as type I interferon antagonists. Among the structural proteins, fusion (F) protein, glyco (G) protein and small hydrophobic (SH) protein constitute the viral surface as glycoproteins, and nucleocapsid (N) protein, phosphoprotein (P), matrix (M) protein, viral polymerase (L), etc. constitute the internal structure of the virus or play important roles in viral replication.

RSV's F protein is an important factor involved in the initial stage of virus entry and fusion with cell membranes, and is known as a major target for vaccines and antiviral drugs due to high antigenicity. However, it is difficult to produce and purify the F protein antigen as a stable prefused protein, and it is difficult to maintain its stability and efficacy.

Therefore, the inventors of the present disclosure aim at solving the instability of the F protein and developing a new type of safe RSV live vaccine.

### DISCLOSURE

### Technical Problem

The present disclosure is directed to developing a live RSV vaccine strain with superior stability by substituting or inserting a heterogeneous F protein with high stability.

The present disclosure is directed to developing a live RSV vaccine strain with superior safety (attenuated) by substituting or inserting a heterogeneous F protein with high stability.

The present disclosure is directed to developing a new recombinant RSV.

The present disclosure is directed to providing a new type of RSV vaccine capable of inducing a defense mechanism against RSV. The present disclosure is directed to providing a new type of vaccine allowing effective operation of the immune system by inhibiting the immune escape mechanism through mutation of the G protein of RSV.

The present disclosure is directed to producing a bivalent vaccine by inserting or substituting a heterogeneous F protein.

### Technical Solution

In an exemplary embodiment, the present disclosure provides a recombinant respiratory syncytial virus (RSV). It provides a recombinant respiratory syncytial virus (RSV) in which the genes encoding the envelope proteins of RSV are rearranged. That is to say, the present disclosure provides a recombinant respiratory syncytial virus wherein a foreign gene is inserted in an RSV gene. In an exemplary embodiment, the present disclosure provides a recombinant respiratory syncytial virus wherein an F protein derived from a virus other than RSV is inserted between the genes encoding the glycoprotein (G protein) and the fusion protein (F protein) of RSV or substituted instead of the F protein of RSV. Specifically, (1) a gene encoding the F protein derived from a heterologous virus belonging to the family Paramyxoviridae or the family Pneumoviridae may be inserted, or (2) a gene encoding the F protein of RSV may be substituted with a gene encoding the F protein derived from a heterologous virus belonging to the family Paramyxoviridae or the family Pneumoviridae. The heterologous virus belonging to the family Paramyxoviridae or the family Pneumoviridae may be one or more virus selected from a group consisting of the genera Aquaparamyxovirus, Avulavirus, Ferlavirus, Hempavirus, Morbillivirus, Respirovirus, Rubulavirus, Metapneumovirus and Orthopneumovirus, specifically one or more virus selected from a group consisting of measles virus and Newcastle disease virus. The recombinant respiratory syncytial virus (RSV) may be a recombinant RSV which is based on the human RSV A virus strain of SEQ ID NO 1 and wherein the genes encoding the envelope proteins of the RSV A virus strain (G protein- and F protein-expressing domains) are rearranged in the recombinant RSV backbone of SEQ ID NO 3. Specifically, it may have cDNA sequences of SEQ ID NOS 4-7. A sequence wherein a restriction enzyme sequence is added to the antigenome of RSV may be represented by SEQ ID NO 2.

In another exemplary embodiment, the present disclosure provides a method for producing an attenuated recombinant RSV using an expression vector including a polynucleotide encoding a recombinant RSV antigenome. In an exemplary embodiment, the recombinant RSV may be produced by expressing a first expression vector including a polynucleotide encoding a recombinant RSV antigenome and a second expression vector including a polynucleotide encoding one or more protein selected from a group consisting of N, P, L and M2-1 proteins together. The polynucleotide encoding a recombinant RSV antigenome includes a polynucleotide encoding a domain of a protein derived from a heterogeneous virus wholly or partially. The polynucleotide encoding a recombinant RSV antigenome included in the first expression vector is any one selected from SEQ ID NOS 4-7. The polynucleotide selected from SEQ ID NOS 4-7 may be a cDNA. The first expression vector may include the polynucleotide encoding a recombinant RSV antigenome in a pCC1 plasmid.

In an exemplary embodiment, the present disclosure provides a recombinant RSV expression vector. The expression vector may include the genes encoding the glycoprotein (G protein) and the fusion protein (F protein) of RSV, wherein a gene encoding the F protein derived from a heterologous virus belonging to the family Paramyxoviridae or the family Pneumoviridae is inserted between the genes encoding the G protein and the F protein, or may include a recombinant RSV gene in which a gene encoding the F protein derived from a heterologous virus belonging to the family Paramyxoviridae or the family Pneumoviridae is substituted instead of a gene encoding the fusion protein (F protein) of RSV. The recombinant RSV gene may be any one selected from SEQ ID NOS 4-7. The recombinant RSV expression vector includes a T7 promoter, a hammerhead ribozyme, a recombinant RSV gene, a hepatitis delta virus ribozyme and a T7 terminator in sequence. The heterologous virus belonging to the family Paramyxoviridae or the family Pneumoviridae may be one or more virus selected from the genera Aquaparamyxovirus, Avulavirus, Ferlavirus, Hempavirus, Morbillivirus, Respirovirus, Rubulavirus, Metapneumovirus and Orthopneumovirus, specifically one or more selected from a group consisting of measles virus and Newcastle disease virus.

In another exemplary embodiment, the present disclosure provides an attenuated RSV live vaccine composition containing the recombinant respiratory syncytial virus (RSV) according to an exemplary embodiment of the present disclosure and a pharmaceutically acceptable carrier. The present disclosure also provides a method for preventing respiratory syncytial virus (RSV) infection by administering the live vaccine composition into the human body.

The present disclosure provides a live RSV strain, which is a recombinant respiratory syncytial virus (RSV) in which a polynucleotide encoding the domain of an F protein derived from a heterologous virus wholly or partially is inserted in the RSV genome. The polynucleotide is i) inserted instead of a polynucleotide encoding the domain of the RSV F protein partially, or ii) inserted in any portion of the polynucleotide constituting the RSV gene. The recombinant RSV of the present disclosure may be used as an RSV vaccine strain and may be used as a vaccine because it exhibits excellent stability and safety.

Hereinafter, the present disclosure is described specifically.

The present disclosure is directed to providing a new type of RSV live vaccine in which a gene encoding the F protein of RSV is substituted with a gene encoding the F protein of a heterogeneous virus, or a gene encoding the F protein of a heterogeneous virus is introduced further.

### DEFINITION OF TERMS

The term “recombinant” may mean that cells with different hereditary characteristics exist together in an organism. Particularly, the term “recombinant” used in the present disclosure refers to the occurrence of new genetic variation as the genetic information (nucleic acid) of different individuals based on one genetic backbone is inserted.

A recombinant RSV refers to an RSV or RSV-like virus derived directly or indirectly or produced from a recombinant expression system or proliferated from subviral particles. The recombinant expression system includes a recombinant expression vector, which includes a functionally linked transcription unit including at least one genetic element or a combination of elements having regulatory functions in the expression of the RSV gene. Examples of the element include a promoter, a structural or coding sequence transcribed to RSV mRNA, or an appropriate initiation or termination sequence.

The term “genome” used in the present disclosure refers to all the base sequences of the genes of an individual, which is a collection of all the genetic information of an organism. For example, the genome of a virus is used to mean all the genetic information of the virus.

The term “gene” used in the present disclosure may be understood as a portion which encodes a protein, which may be a DNA or an RNA.

The term “introduction” used in the present disclosure refers to addition of a new element to the original elements. Mutation of a gene, such as deletion, substitution, addition, etc., may be induced by the introduction.

For example, the expression that “a new gene was introduced to a genome” used in the present disclosure may mean that a portion of the whole gene base sequence of an individual was substituted or inserted with a gene derived from a new individual. As a result of the introduction, the original genome base sequence (e.g., backbone) of the individual may become longer or shorter, or may remain unchanged.

The term “vaccine strain” used in the present disclosure refers to a population of viruses isolated for use as a vaccine.

The term “live attenuated vaccine” used in the present disclosure refers to a vaccine prepared from attenuated respiratory syncytial virus (RSV), which has attenuated virulence of a pathogen so as not to cause a disease in a host but still keeps the pathogen viable.

The term “antigenome” used in the present disclosure refers to a complementary (+)-sense polynucleotide molecule that functions as a template for synthesis of a progeny RSV genome. The native RSV genome generally includes (−)-sense polynucleotide molecules, which encode viral proteins, such as glycoproteins (G), fusion proteins (F), N, P, etc. through complementary viral mRNAs. The attenuated RSV live vaccine strain may be prepared through recombination of cDNAs encoding the RSV genome or antigenome.

The term “gene encoding a protein” or “protein-encoding gene” used in the present disclosure refers to a gene which produces a protein.

In an exemplary embodiment, the present disclosure provides a live RSV vaccine strain with superior stability and safety in which an F protein of a heterologous virus is inserted into the RSV genome, or the F protein of RSV is substituted with an F protein of a heterologous virus.

In an exemplary embodiment, the present disclosure provides a recombinant respiratory syncytial virus (RSV) vaccine strain in which a polynucleotide encoding some domains of an F protein derived from a heterologous virus is inserted into the RSV genome.

The polynucleotide may be i) inserted instead of a polynucleotide encoding some domains of the RSV F protein, or ii) inserted between the RSV F protein and the polynucleotide encoding the G protein domain.

FIG. 2 shows substitution of the polynucleotide encoding some domains of the F protein of RSV by a polynucleotide encoding some domains of an F protein derived from a foreign virus.

FIG. 3 shows insertion of some domains of an F protein derived from a foreign virus into the polynucleotide encoding the F protein of RSV.

The heterologous virus may be substituted with a gene encoding the F protein derived from a heterologous virus belonging to the family Paramyxoviridae or the family Pneumoviridae. The heterologous virus belonging to the family Paramyxoviridae or the family Pneumoviridae may be one or more selected from a group consisting of the genera Aquaparamyxovirus, Avulavirus, Ferlavirus, Hempavirus, Morbillivirus, Respirovirus, Rubulavirus, Metapneumovirus and Orthopneumovirus, specifically one or more selected from a group consisting of measles virus and Newcastle disease virus. In particular, the measles virus and Newcastle disease virus may improve the stability and safety of the live vaccine virus strain.

Specifically, the RSV genome, which is the basis of the present disclosure, may be a wild-type human RSV A virus strain of SEQ ID NO 1. Although various RSV strains are available, including RSV A, RSV B, HRSV A, HRSV B, BRSV and avian RSV strains, the RSV backbone to which a foreign gene is inserted may be specifically an RSV A virus strain. When a foreign gene is introduced into the RSV A virus strain, the produced recombinant virus may have superior stability and safety and low mutation rate.

In an exemplary embodiment, the present disclosure provides a method for producing an attenuated recombinant RSV by expressing a first expression vector including a polynucleotide encoding a recombinant RSV antigenome and a second expression vector including a polynucleotide encoding one or more protein selected from a group consisting of N, P, L and M2-1 proteins together. As the first expression vector, pCC1, pBR322, pUC, pcDNA3.1, etc. may be used. However, when considering size, virulence, etc., pCC1 may be specifically used for the purpose of the present disclosure. As the second expression vector, a pCI neo vector may be used, although not being limited thereto.

RSV is a negative-sense RNA virus and requires a helper gene for viral production and gene synthesis through reverse genetics in vitro. The polynucleotide encoding the recombinant RSV antigenome or the polynucleotide encoding the N, P, L or M2-1 protein may be introduced into a cell by transfection, electroporation, mechanical insertion or transduction. Viral infection may occur in such cells as Vero cells, Hep 2 cells, MRC 5 cells, etc. Vero cells may be favorable for production of the recombinant RSV strain of the present disclosure.

As the method for introducing the polynucleotide encoding the recombinant RSV antigenome or the polynucleotide encoding the N, P, L or M2-1 protein, methods commonly used in the art may be used without special limitation.

In another exemplary embodiment, an RSV antigenome RNA may be synthesized in vitro and then transfected into a cell expressing the RSV protein.

The polynucleotide encoding a recombinant RSV antigenome may be included in a first expression vector, and the helper gene may be included in a vector (second expression vector) which is different from the first expression vector. Alternatively, they may be included in the same vector.

The polynucleotide encoding a recombinant RSV antigenome included in the first expression vector may include one or more polynucleotide selected from SEQ ID NOS 4-7, and the polynucleotide selected from SEQ ID NOS 4-7 may be a cDNA.

The first expression vector may include the polynucleotide encoding a recombinant RSV antigenome in a pCC1 plasmid. For the purpose of easier combination, the vector may be modified by inducing mutation, modifying the restriction enzyme site or inserting a polylinker including a modified restriction enzyme site.

The pCC1 plasmid of the present disclosure may stabilize the recombinant RSV antigenome.

Specifically, the first expression vector including the recombinant RSV antigenome may be an expression vector including the cDNA of a polynucleotide, including a T7 promoter, a hammerhead ribozyme, an RSV antigenome, a hepatitis delta virus ribozyme and a T7 terminator.

The RSV antigenome includes the F protein of measles virus, Newcastle disease virus and parainfluenza virus (PIV), and a polynucleotide encoding the transmembrane (TM) and cytoplasmic tail (CT) domains of the F protein may be one derived from RSV or one derived from the above-described viruses. In an exemplary embodiment, the present disclosure provides an attenuated RSV vaccine strain produced by the method described above.

In an exemplary embodiment, the present disclosure provides an RSV live vaccine including the RSV strain of the present disclosure and a pharmaceutically acceptable carrier.

The recombinant RSV vaccine strain produced according to an exemplary embodiment of the present disclosure may be used as a vaccine as it is, after being freeze-dried or after being mixed in a liquid.

The vaccine composition of the present disclosure contains any pharmaceutical substance which does not cause an immune response harmful to a vertebrate receiving the composition on its own, and contains a pharmaceutically acceptable carrier including any appropriate diluent or excipient that can be administered together with the recombinant RSV vaccine strain without unreasonable toxicity. The term “pharmaceutically acceptable” means listed in the United States Pharmacopeia, European Pharmacopeia or other generally recognized pharmacopeia for use in vertebrates, more specifically in human. The RSV F antigen of the present disclosure is administered with an effective amount (defined above) sufficient for stimulating an immune response in response to one or more strain of RSV. The composition may be used as a vaccine and/or immunogenic composition for inducing a protective immune response in vertebrates.

In a non-limiting exemplary embodiment, the concentration of the recombinant RSV antigen included in the vaccine is at least about 10 μg/mL, about 20 μg/mL, about 30 μg/mL, about 40 μg/mL, about 50 μg/mL, about 60 μg/mL, about 100 μg/mL, about 200 μg/mL or about 500 μg/mL. In a specific exemplary embodiment, the concentration of the immunogen is from about 10 μg/mL to about 1 mg/mL, from about 20 μg/mL to about 500 μg/mL, from about 30 μg/mL to about 100 μg/mL, or from about 30 μg/mL to about 50 μg/mL. In another exemplary embodiment, the concentration of the immunogen may be 10-200 μg/mL.

The vaccine or immunogenic composition of the present disclosure may be administered to an animal to induce an immune response against RSV. In an exemplary embodiment, the animal is human. Generally, the administration dosage may be may be adjusted on the basis of, for example, age, physical condition, body weight, diet, administration time and other clinical factors. Accordingly, the present disclosure includes a method for preparing a vaccine or immunogenic composition inducing substantial immunity against infection of a subject or at least one symptom thereof, which includes a step of adding an effective amount of an immunogen in the composition. Although it is preferred that the substantial immunity is induced by a single administration dosage, an additional administration dosage may be administered through the same or different route to achieve desired effect. For newborns and infants, a number of administrations may be necessary, for example, to induce a sufficient level of immunity. The administration may be continued over the infancy if it is necessary to provide a sufficient level of protection against infection. In an exemplary embodiment, a method for inducing substantial immunity against viral infection or at least one symptom thereof in a subject includes a step of administering an effective amount of the RSV recombinant F protein, a fragment thereof or an aggregate thereof at least once. The vaccine and/or immunogen composition may be administered by parenteral (e.g., endothelial, intramuscular, intravenous or subcutaneous), epidural or mucosal (e.g., nasal, oral, pulmonary or suppository) administration, although not being limited thereto. In a specific exemplary embodiment, the composition is administered intramuscularly, intravenously, subcutaneously, orally or endothelially. The composition may be administered via any convenient route, for example, by infusion or bolus injection or absorption through the epithelium or mucous membrane (e.g., oral, colonic, conjunctival, nasopharyngeal, oropharyngeal, vaginal, urinary tract, bladder or intestinal mucosa) and may be administered together with another biologically active substance. The administration may be systemic or topical. The preventive vaccine composition may be administered via subcutaneous or intramuscular injection using a needle and a syringe or may be administered systemically using a needle-free injection device. Optionally, the vaccine composition may be administered nasally into the upper airway in the form of droplets or large-particle aerosols (larger than about 10 μm) through spraying. Whereas some of the above-mentioned pathways cause immune response at random sites, the nasal administration provides enhanced effect of inducing mucosal immunity at the virus-infected site. In another exemplary embodiment, the vaccine and/or immunogenic composition may be administered to target a mucosal tissue without inducing an immune response at the immunization site. The site of administration is not limited.

In an exemplary embodiment, the present disclosure provides a method for preventing respiratory syncytial virus (RSV) infection by administering the RSV vaccine strain of the present disclosure into the human body.

### Advantageous Effects

The present disclosure provides a live RSV vaccine strain with superior stability in which a heterogeneous F protein with high stability is substituted or inserted. It provides a new recombinant RSV strain which has an antibody titer comparable to that of wild-type RSV while solving the unsafety and instability of common live vaccines.

The present disclosure provides a live RSV vaccine strain with superior safety in which a heterogeneous F protein with high stability is substituted or inserted.

The present disclosure provides a new type of RSV vaccine capable of inducing a defense mechanism against RSV.

The present disclosure provides a new type of vaccine allowing effective operation of the immune system by inhibiting the immune escape mechanism through mutation of the G protein of RSV.

The present disclosure allows production of a bivalent vaccine by inserting or substituting a heterogeneous F protein.

The present disclosure provides a new recombinant RSV.

### DESCRIPTION OF DRAWINGS

FIG. 1 is a schematic diagram of the RSV genome.

FIG. 2 schematically shows the cleavage of the F protein region of the RSV genome (arrows indicate the cleavage sites) and the substitution with an F protein of a heterogeneous virus (e.g., NDV).

FIG. 3 schematically shows the addition of an F protein of a heterogeneous virus (e.g., MV) between the G protein and the F protein of the RSV genome.

FIG. 4 shows the cleavage of the MluI and ApaI sites from the backbone of SEQ ID NO 3 and insertion of an F protein derived from a foreign virus. In the final construct \textcircled{3}, G GE indicates the gene end region of the G protein, F GS indicates the gene starting region of the F protein, and F GE indicates the gene end region of the F protein. The F protein of MV or NDV is inserted, but the T and C domains of the F protein are those of RSV.

FIG. 5 shows the cleavage of the MluI and MluI sites from the backbone of SEQ ID NO 3 and insertion of an F protein derived from a foreign virus. The MluI and MluI cleavage sites are located between the G protein and the F protein of RSV. In the final construct \textcircled{3}, G GE indicates the gene end region of the G protein, F GS indicates the gene starting region of the F protein, and F GE indicates the gene end region of the F protein. The F protein of MV or NDVF is inserted, but the T and C domains of the F protein are those of RSV.

FIG. 6 schematically shows the cloning of the cDNA of the recombinant RSV of the present disclosure virus into a vector.

FIG. 7 shows the attenuation of recombinant RSV strains for different cells (HEp2 cells, Vero cells and MRCS cells) through comparison of proliferation rate and proliferation titer. The proliferation rate and titer were decreased for the vaccine strains as compared to the wild-type RSV.

FIG. 8 shows the attenuation of recombinant RSV strains in mouse through comparison of proliferation rate and proliferation titer. The proliferation rate and titer were decreased for the vaccine strains as compared to the wild-type RSV.

FIG. 9 shows the stability of recombinant RSV strains depending on temperature. The recombinant RSV strains showed improved thermal stability as compared to the wild-type RSV at all the temperatures of 4° C., 37° C. and 60° C.

FIG. 10 shows a result of investigating total antibody titer and neutralizing antibody titer in a mouse immunity test of recombinant RSV. Sufficient antigen specificity and neutralizing antibodies were induced in all groups except Mock. However, the formalin-inactivated wild-type virus G7 showed relatively very low total antibody titer and neutralizing antibody titer. The RSV backbone was denoted as wtRSVA_TH10654, RSV backbone-MV F substitution as cRSVA_MVF_S, RSV backbone-NDV F substitution as cRSVA_NDVF_S, RSV backbone-MV F insertion as cRSVA_MVF_A, and RSV backbone-NDV F insertion as cRSVA_NDVF_A.

In FIG. 10, Mock was denoted as G1, WtRSVA as G2, cRSVA_MVF_S as G3, cRSVA_NDVF_S as G4, cRSVA_MVF_A as G5, cRSVA_NDVF_A as G6, and FIwtRSVA as G7.

FIG. 11 shows a result of measuring defensive power after immunization with recombinant RSV by challenging with wild-type RSV. Sufficient protective immunity was induced in all groups except Mock. However, the formalin-inactivated wild-type virus G7 exhibited slightly insufficient protective immunity.

FIG. 12 shows a test result of immunogenicity and neutralizing antibody titer for NDV.

FIG. 13 shows a test result of immunogenicity and neutralizing antibody titer for MV immunogenic.

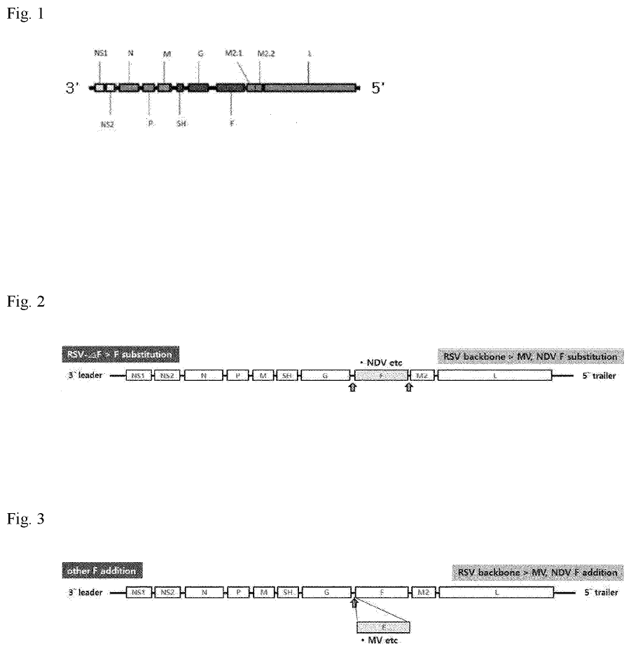

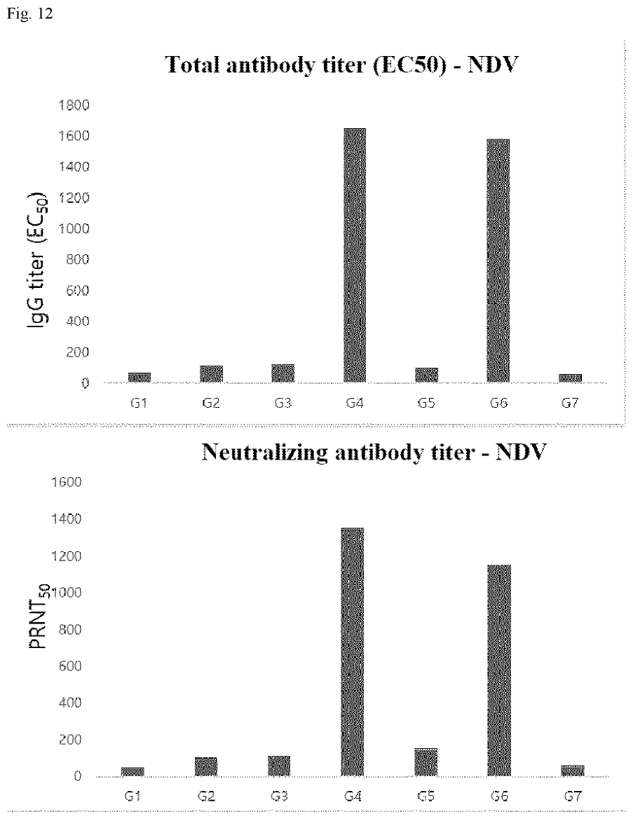

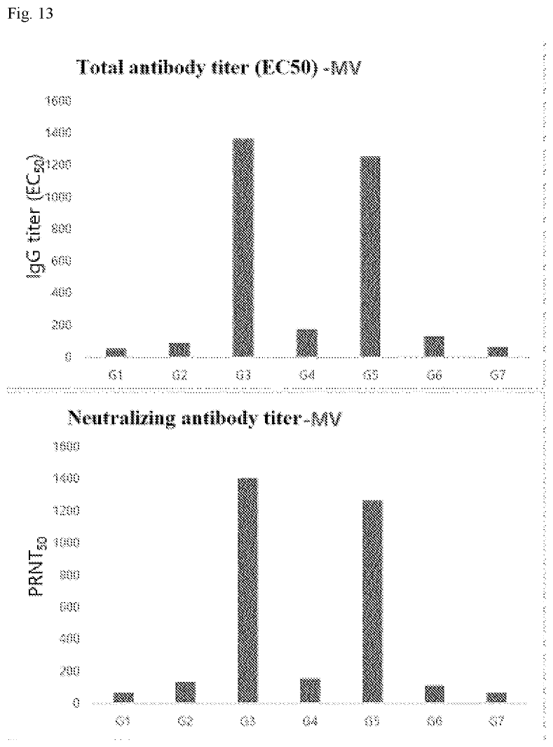

In FIGS. 12 and 13, Mock was denoted as G1, WtRSVA as G2, cRSVA_MVF_S as G3, cRSVA_NDVF_S as G4, cRSVA_MVF_A as G5, cRSVA_NDVF_A as G6, and FIwtRSVA as G7.

### MODE FOR DISCLOSURE

Hereinafter, the present disclosure is described in detail through examples, etc. to help understanding. However, the examples according to the present disclosure may be changed into various other forms and it should not be interpreted that the scope of the present disclosure is limited by the following examples. The examples of the present disclosure are provided such that the present disclosure is more completely described to those having average knowledge in the art.

1. Preparation of Wild-Type RSV a Virus Strain

A wild-type RSV A virus strain having the following information was prepared described below.
Definition: human respiratory syncytial virus strain RSVA/TH_10654/complete genome
Accession No.: KU950464.1
Length: 15232 bp
Host: Homo sapiens/female/12 weeks
Collection date: 19 Feb. 2014
Country: USA
Subtype: RSV A

The strain is represented by SEQ ID NO 1.

2. Preparation of F Protein Donor Virus

Measles virus (MV) and Newcastle disease virus (NDV) were selected as F protein donors.

3. Preparation of cDNA Encoding RSV Antigenome

A polynucleotide encoding the F protein of the RSV antigenome was constructed by inserting or substituting a polynucleotide encoding an F protein derived from a heterologous virus to backbone construct A described below.

(1) Designing of backbone construct A (SEQ ID NO 3)

The gene sequence was T7 promoter—hammerhead ribozyme—RSV antigenome—hepatitis delta virus ribozyme—T7 terminator.

A T7 promoter sequence (TAATACGACTCACTATAGG) was inserted to the 5′-end. After the T7 promoter sequence, a hammerhead ribozyme sequence (T TTTTTCGCGT CTGATGAGGC CGTTAGGCCG AAACTCCTCT CCGGAGTC) was inserted. After the hammerhead ribozyme sequence, a wild-type RSV antigenome sequence was inserted and the following mutation was induced.

An AscI restriction enzyme sequence was produced by inserting GCGCGCC between 77 nt and 78 nt on the basis of the antigenome sequence of wild-type RSV (Although the AscI restriction enzyme sequence was GGCGCGCC <8 bp>, only GCGCGCC <7 bp> was inserted beceause the 77 nt sequence was G.)

1) CCTGCAGG (SbfI restriction enzyme sequence) was inserted between 1079 nt and 1080 nt.

2) GGCCGGCC (FseI restriction enzyme sequence) was inserted between 4600 nt and 4601 nt.

3) The base ATG(M) at 4800 nt and 4802 nt was substituted with ATT(I).

4) ACGCGT (MluI restriction enzyme sequence) was inserted between 5639 nt and 5640 nt.

5) GGGCCC (ApaI restriction enzyme sequence) was inserted between 7595 nt and 7596 nt.

6) After the wild-type RSV antigenome sequence, a hepatitis delta virus ribozyme sequence (GGCCGGCATGGTCCCAGCCTCCTCGCTGGCGCCGGCTGGGCAACATGCTTCGGC ATGGCGAATGGGAC) was inserted.

7) After the hepatitis delta virus ribozyme sequence, a T7 terminator sequence (TAGCATAACCCCTTGGGGCCTCTAAACGGGTCTTGAGGGGTTTTTTG) was inserted.

8) The ACGCGAAAAAATGCGTACAAC sequence was inserted at the 5′-end, and the GTTTTTGACACTTTTTTTCTCGT sequence was inserted at the 3′-end. The ACGCGAAAAAATGCGTACAAC inserted at the 5′-end was denoted as a 3′ leader, and the GTTTTTGACACTTTTTTTCTCGT inserted at the 3′-end was denoted as a 5′ trailer.

The designed gene (SEQ ID NO 3) was synthesized in vitro and then cloned into a pCC1 vector.

A point mutation (M48I) of the G protein sequence was introduced.

(2) Designing of Measles virus F-substituted construct B (SEQ ID NO 4)

On the basis of the backbone construct A, the F protein gene sequence was removed and a corresponding measles virus F gene sequence was inserted.

The antigenome data of the Edmonston strain (Moraten vaccine) (Accession No. AF266287.1), which is a measles virus strain, were used.

The F protein gene of the Edmonston strain antigenome corresponds to sequences 5449-7110. Among them, sequences 5449-6939 excluding the transmembrane region and cytoplasmic tail region sequences were used. As the transmembrane region and cytoplasmic tail region sequences, the sequences of the wild-type human respiratory syncytial virus strain RSVA/TH_10654 were used. As 5′ UTR and 3′ UTR, the UTR sequences of the F protein gene of the wild-type human respiratory syncytial virus strain RSVA/TH_10654 were used.

After cleaving the backbone construct A with MluI and ApaI restriction enzymes, the designed measles virus F gene was inserted.

(3) Designing of Newcastle disease virus F-substituted construct C (SEQ ID NO 5)

After removing the F protein gene sequence from the backbone construct A, the Newcastle disease virus F gene sequence corresponding thereto was inserted.

The antigenome data of the 1-2 strain (Accession No. AY935499), which is a Newcastle disease virus, were used.

The F protein gene of the 1-2 strain genome corresponds to sequences 4544-6205 sequence. Among them, sequences 4544-6043 excluding the transmembrane region and cytoplasmic tail region sequences were used. As the transmembrane region and cytoplasmic tail region sequences, the sequences of the wild-type human respiratory syncytial virus strain RSVA/TH_10654 were used. As 5′ UTR and 3′ UTR, the UTR sequences of the F protein gene of the wild-type human respiratory syncytial virus strain RSVA/TH_10654 were used.

After cleaving the backbone construct A with MluI and ApaI restriction enzymes, the Newcastle measles virus F gene was inserted.

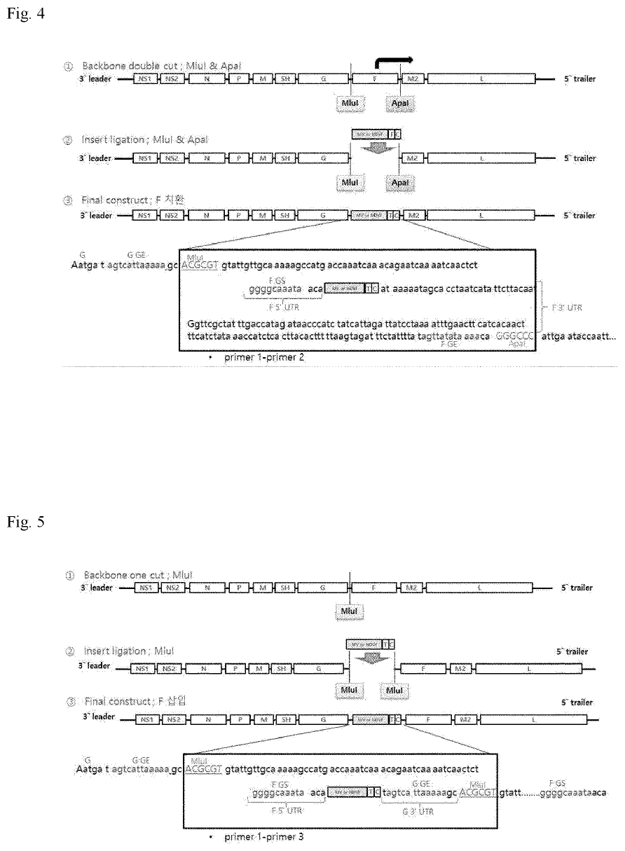

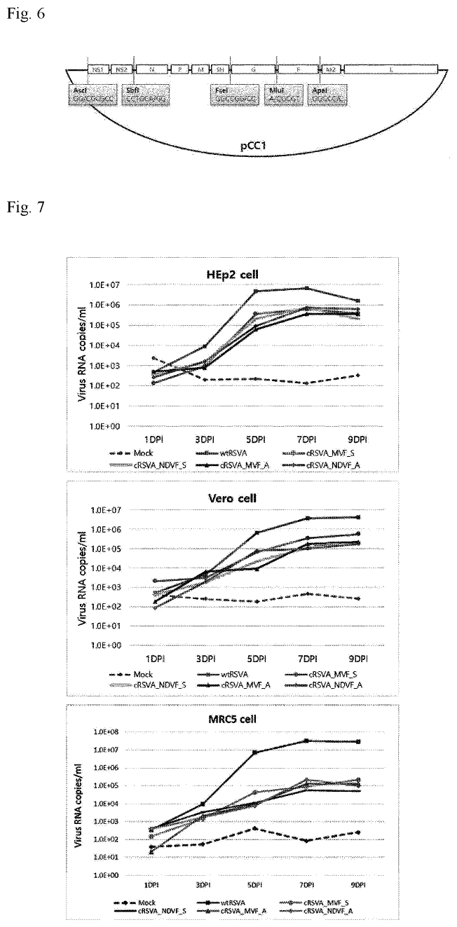

As seen from FIG. 4, the constructs B and C recognize the restriction enzymes MluI/ApaI and amplify the inserts using primers 1 and 2. The primer 1 (forward) was 5′ ATAT ACGCGT gtattgttgcaaaaagccatg, and the primer 2 (reverse) was 5′ ATAT GGGCCC tgttttatataactataaaatagaa.

(4) Designing of Measles virus F-inserted construct D (SEQ ID NO 6)

The measles virus F gene sequence was inserted between the G protein and F protein genes of the backbone construct A.

The antigenome data of the Edmonston strain (Moraten vaccine) (Accession No. AF266287.1), which is a measles virus strain, were used.

The F protein gene of the Edmonston strain antigenome corresponds to sequences 5449-7110. Among them, sequences 5449-6939 excluding the transmembrane region and cytoplasmic tail region sequences were used. As the transmembrane region and cytoplasmic tail region sequences, the sequences of the wild-type human respiratory syncytial virus strain RSVA/TH_10654 were used. As 5′ UTR, the 5′ UTR sequence of the F protein gene of the wild-type human respiratory syncytial virus strain RSVA/TH_10654 was used. As 3′ UTR, the 3′ UTR sequence of the G protein gene of the wild-type human respiratory syncytial virus strain RSVA/TH_10654 was used.

After cleaving the backbone construct A with the MluI restriction enzyme, the designed measles virus F gene was inserted.

(5) Designing of Newcastle disease virus F-inserted construct E (SEQ ID NO 7)

The Newcastle disease virus F gene sequence was inserted between the G protein and F protein genes of the backbone construct A.

The gene data of the 1-2 strain (Accession No. AY935499.1), which is a Newcastle disease virus strain, were used.

The F protein gene of the I-2 strain genome corresponds to sequences 4544-6205 sequence. Among them, sequences 4544-6043 excluding the transmembrane region and cytoplasmic tail region sequences were used. As the transmembrane region and cytoplasmic tail region sequences, the sequences of the wild-type human respiratory syncytial virus strain RSVA/TH_10654 were used. As 5′ UTR, the 5′ UTR sequence of the F protein gene of the wild-type human respiratory syncytial virus strain RSVA/TH_10654 was used. As 3′ UTR, the 3′ UTR sequence of the G protein gene of the wild-type human respiratory syncytial virus strain RSVA/TH_10654 was used.

After cleaving the backbone construct A with the MluI restriction enzyme, the designed Newcastle disease virus F gene was inserted.

As seen from FIG. 5, the heterologous virus gene was inserted to the region recognized by the restriction enzyme MluI and the insert was amplified using primers 1 and 3. The primer 1 (forward) was 5′ ATAT ACGCGT gtattgttgcaaaaagccatg, and the primer 3 (reverse) was 5′ ATAT ACGCGT gclllltaatgacta tcagttactaaatgcaatat.

4. Designing of Four Helper Genes

RSV is a negative-sense RNA virus. While producing the virus in vitro through reverse genetics, helper genes (genes encoding N, P, L and M2-1 proteins of RSV) necessary for gene synthesis were added together.

(1) The N protein gene clones the sequences 1119-2294 of the wild-type human respiratory syncytial virus strain RSVA/TH_10654 antigenome into a pCI neo vector using the restriction enzymes XhoI and MluI.

(2) The P protein gene clones the sequences 2326-3051 of the wild-type human respiratory syncytial virus strain RSVA/TH_10654 antigenome into a pCI neo vector using the restriction enzymes XhoI and MluI.

(3) The M2-1 protein gene clones the sequences 7647-8231 of the wild-type human respiratory syncytial virus strain RSVA/TH_10654 antigenome into a pCI neo vector using the restriction enzymes XhoI and MluI.

(4) The L protein gene clones the sequences 8539-15036 of the wild-type human respiratory syncytial virus strain RSVA/TH_10654 antigenome into a pCI neo vector using the restriction enzymes XhoI and MluI.

The N protein gene is represented by SEQ ID NO 8, the P protein gene by SEQ ID NO 9, the M2-1 protein gene by SEQ ID NO 10, and the L protein gene by SEQ ID NO 11.

5. Virus Rescue

BHK cells were prepared on a 6-well plate at a concentration of 4×10⁵ cell/well.

Next day, after mixing six plasmids, i.e., 2 μg of a T7 polymerase expression vector, 2 μg of a full-length RSV antigenome vector and 1 μg of four helper gene vectors (N, P, M2-1 and L), with 10 μL of Lipofectamine 3000, and reacting for 10 minutes, the mixture was transfected to BHK cells. The mixture was added dropwise for uniform application to the cells and then shaken 2-3 times to ensure mixing.

After culturing the cells for 5 days in a CO₂ incubator at 37° C., the culture was recovered and then transfected (1 mL each) to Vero cells (3×10⁵/well) on a freshly prepared 6-well plate. A virus solution was obtained by culturing again for 5 days. One recombinant wild-type RSV and four RSV vaccine strains were produced in this way.

This technology is an experimental method commonly used by researchers for RSV rescue and there is no special technical limitation. In this experiment, the literatures “BAC-Based Recovery of Recombinant Respiratory Syncytial Virus (RSV); Reverse Genetics of RNA Viruses, pp. 111-124” and “RNA Virus Reverse Genetics and Vaccine Design; Viruses, 2014, 6, 2531-2550” were referred to. After recovering the culture, the virus was detected through IFA, RT-PCR and gene sequencing.

\textcircled{1} RSV backbone strain: wtRSVA_TH10654 (wild-type control)

\textcircled{2} RSV backbone strain—MV F substitution: cRSVA_MVF_S (mutant virus)

\textcircled{3} RSV backbone strain—NDV F substitution: cRSVA_NDVF_S (mutant virus)

\textcircled{4} RSV backbone strain—MV F insertion: cRSVA_MVF_A (mutant virus)

\textcircled{5} RSV backbone strain—NDV F insertion: cRSVA_NDVF_A (mutant virus)

6. In Vitro Attenuation Test

After infecting Vero cells, HEp2 cells and MRC-5 cells with 0.1 MOI of the five strains, the cells were cultured for 9 days. The virus culture was collected with 1-day intervals and viral titer was analyzed by q-PCR.

As shown in FIG. 7, it was confirmed that the mutant viral vaccine strains were attenuated, with decreased proliferation rate and proliferation titer as compared to the wild-type virus. Through this, it can be seen that the recombinant viruses can be used as live vaccine strains. In the figure, DPI stands for days post-infection.

7. In Vivo Attenuation Test

The five strains of viruses were administered to BALB/c mouse at concentrations of 10¹-10⁷ pfu/mouse via the IN route. Viremia was detected by q-PCR in the blood on days 1-7, which confirms attenuation.

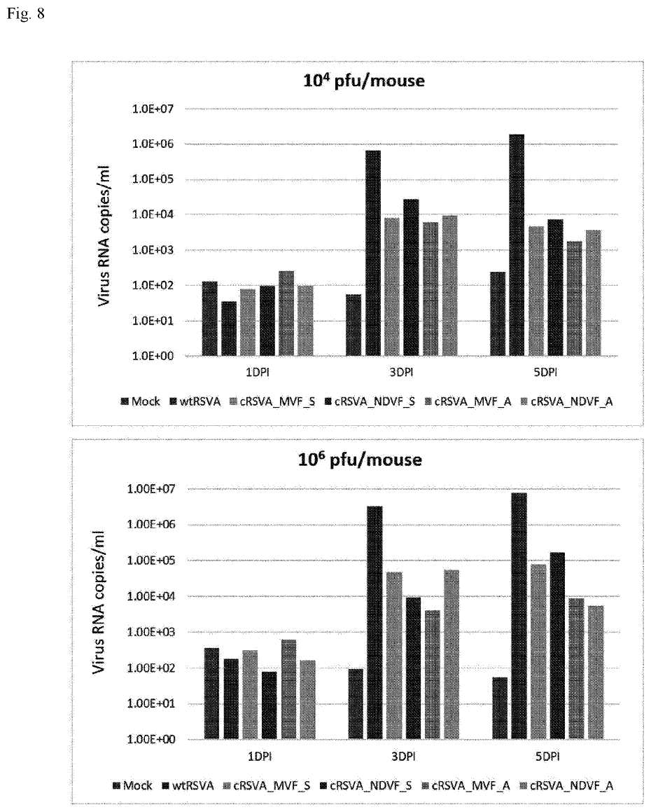

The result is shown in FIG. 8. As can be seen from FIG. 8, it was confirmed that the viruses were attenuated after being vaccinated in vivo as compared to the wild-type RSV A virus. Through this, it was confirmed that the recombinant RSVs can be used as new virus strains.

8. Virus Stability Test

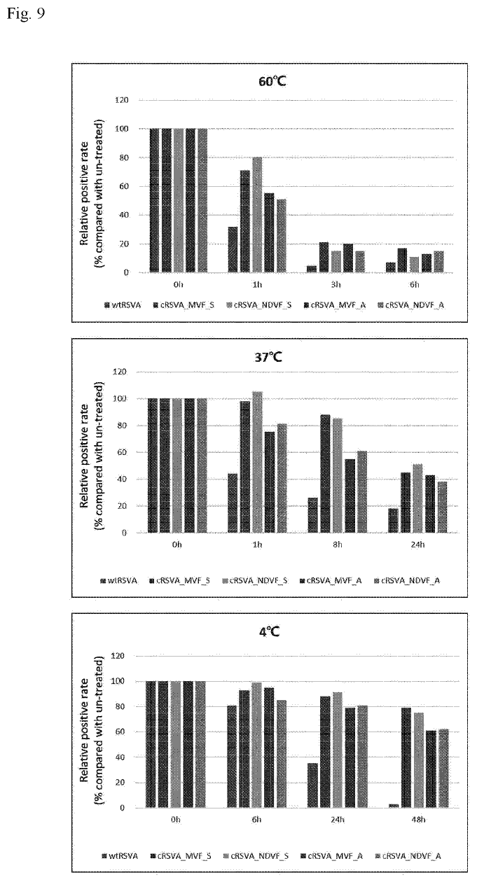

The viability of the five strains of viruses with time was investigated at 60° C., 37° C. and 4° C. Live viral titer in the sample was analyzed through plaque assay using HEp2 cells. As a result, it was confirmed that the stability of the mutant viruses was improved over the wild type under all temperature conditions. The stability of isolated RSV is decreased greatly. However, as can be seen from FIG. 9, the recombinant RSVs exhibited superior stability as compared to the wild-type virus strain at high temperature, human body temperature and low temperature.

9. Immunogenicity Test

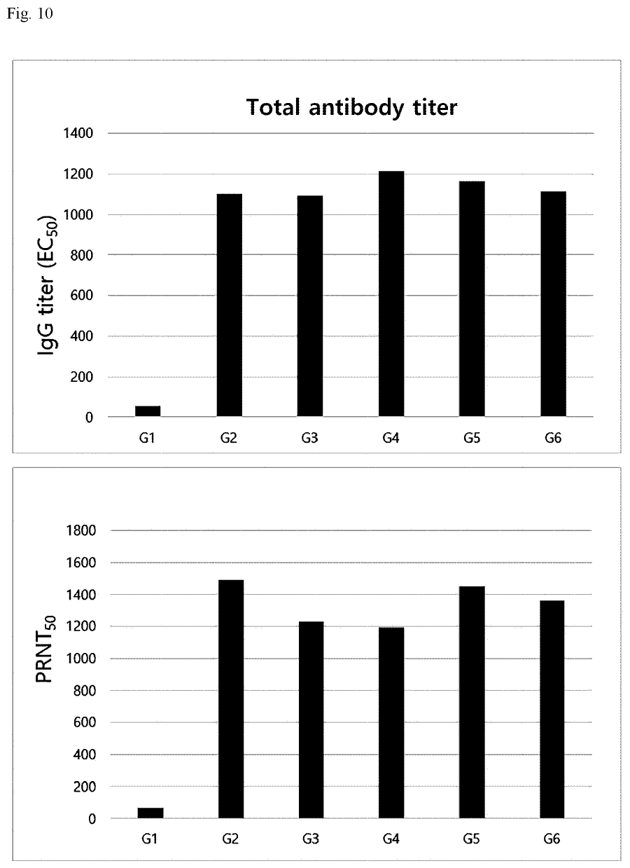

Mouse IM was immunized twice with each of Mock, wtRSVA, cRSVA_MVF_S, cRSVA_NDVF_S, cRSVA_MVF_A and cRSVA_NDVF_A at a concentration of 1×10⁵ pfu/mouse. 2 weeks later, after isolating mouse serum, total antibody titer and neutralizing antibody titer were measured by ELISA and plaque reduction neutralization test. The result is shown in FIG. 10. It was confirmed that RSV antigen-specific IgG antibodies and viral infection-neutralizing antibodies were produced sufficiently in all groups except Mock. Mock was denoted as G1, WtRSVA as G2, cRSVA_MVF_S as G3, cRSVA_NDVF_S as G4, cRSVA_MVF_A as G5, cRSVA_NDVF_A as G6, and FIwtRSVA as G7. It can be seen that the recombinant RSV strains produced antibodies at similar levels to the wild-type RSV, and shoed much higher antibody titers as compared to the inactivated G7. From the result of FIG. 10, it was confirmed that the recombinant RSV strains can be used as excellent virus strains because they exhibit superior stability while producing antibodies at similar levels to the wild-type virus.

10. Immunization Test

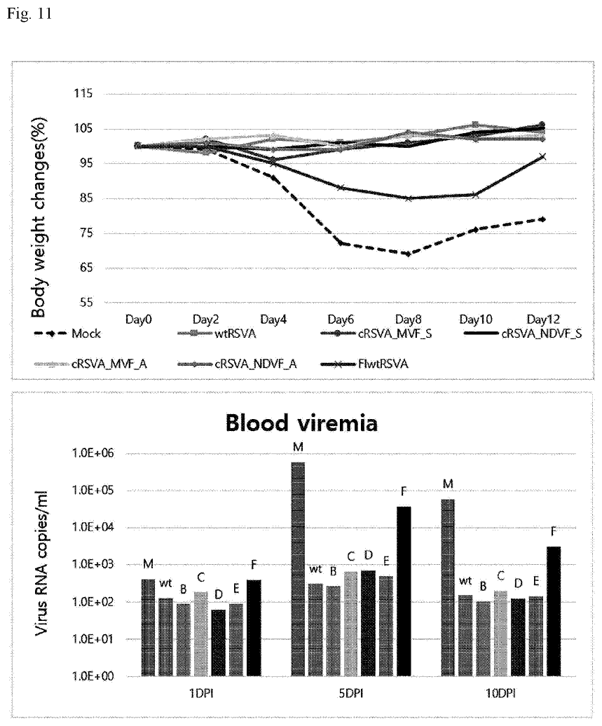

Mouse IM was immunized twice with each of the five strains of viruses at a concentration of 10⁵ pfu/mouse with a 2-week interval. 2 weeks after the immunization, the wild-type RSV was challenged into the nasal cavity of the mouse at a concentration of 2×10⁶ pfu/mouse. On days 1-12, viremia in blood was detected by q-PCR and the change in body weight was measured. The result is shown in FIG. 11. It was confirmed that viral infection was inhibited effectively in all the immunized groups. In particular, the level of inhibition of infection was similar to that of the wild-type virus strain, and the viral infection in blood was remarkably lower as compared to the inactivated virus strain.

11. Immunogenicity Test for MV and NDV

Total antibody titer and neutralizing antibody titer for NDV and MV antigens were measured in a manner similar to 9 by using the same serum. For measurement of total antibody titer, ELISA was conducted after coating with each of MV and DNV antigens. For measurement of neutralizing antibody titer, the concentrations that inhibits MV and NDV infections were measured by plaque inhibition assay. As shown in FIG. 12 and FIG. 13, enough antigen-specific antibody titer and virus neutralizing antibody titer were attained only for the groups having the F antigens of MV and NDV. In contrast, the groups to which the heterogeneous F was not inserted did not produce the antibodies for each antigen and neutralizing antibodies were not induced either.

1. A recombinant respiratory syncytial virus (RSV) in which genes encoding the envelope proteins of RSV are rearranged, wherein
a gene encoding the F protein derived from a heterologous virus belonging to the family Paramyxoviridae or the family Pneumoviridae is inserted between the genes encoding the glycoprotein (G protein) and the fusion protein (F protein) of RSV, or
a gene encoding the F protein of RSV is substituted with a gene encoding the F protein derived from a heterologous virus belonging to the family Paramyxoviridae or the family Pneumoviridae.

2. The recombinant respiratory syncytial virus (RSV) according to claim 1, wherein the heterologous virus belonging to the family Paramyxoviridae or the family Pneumoviridae is one or more virus selected from a group consisting of the genera Aquaparamyxovirus, Avulavirus, Ferlavirus, Henipavirus, Morbillivirus, Respirovirus, Rubulavirus, Metapneumovirus and Orthopneumovirus.

3. The recombinant respiratory syncytial virus (RSV) according to claim 1, wherein the heterologous virus is one or more virus selected from a group consisting of measles virus and Newcastle disease virus.

4. The recombinant respiratory syncytial virus (RSV) according to claim 1, wherein the recombinant respiratory syncytial virus (RSV) is one in which the genes encoding the envelope proteins of the wild-type human RSV A virus strain of SEQ ID NO 1 are rearranged.

5. A method for producing an attenuated recombinant RSV by expressing a first expression vector comprising a polynucleotide encoding a recombinant RSV antigenome and a second expression vector comprising a polynucleotide encoding one or more protein selected from a group consisting of N, P, L and M2-1 proteins, wherein the polynucleotide encoding a recombinant RSV antigenome comprises a polynucleotide encoding a domain of a protein derived from a heterogeneous virus wholly or partially.

6. The method according to claim 5, wherein the polynucleotide encoding a recombinant RSV antigenome comprised in the first expression vector is any one selected from SEQ ID NOS 4-7.

7. The method according to claim 6, wherein the polynucleotide selected from SEQ ID NOS 4-7 is a cDNA.

8. The method according to claim 5, wherein the first expression vector comprises the polynucleotide encoding a recombinant RSV antigenome in a pCC1 plasmid.

9. The method according to claim 5, wherein the protein derived from a heterogeneous virus of the polynucleotide encoding a recombinant RSV antigenome is an F protein.

10. A recombinant RSV expression vector,
comprising the genes encoding the glycoprotein (G protein) and the fusion protein (F protein) of RSV, wherein a gene encoding the F protein derived from a heterologous virus belonging to the family Paramyxoviridae or the family Pneumoviridae is inserted between the genes encoding the G protein and the F protein, or
comprising a recombinant RSV gene in which a gene encoding the F protein derived from a heterologous virus belonging to the family Paramyxoviridae or the family Pneumoviridae is substituted instead of a gene encoding the fusion protein (F protein) of RSV.

11. The recombinant RSV expression vector according to claim 10, wherein the recombinant RSV gene is any one selected from SEQ ID NOS 4-7.

12. The recombinant RSV expression vector according to claim 10, wherein the recombinant RSV expression vector comprises a T7 promoter, a hammerhead ribozyme, a recombinant RSV gene, a hepatitis delta virus ribozyme and a T7 terminator in sequence.

13. The recombinant RSV expression vector according to claim 10, wherein the heterologous virus belonging to the family Paramyxoviridae or the family Pneumoviridae is one or more selected from a group consisting of measles virus and Newcastle disease virus.

14. An attenuated RSV live vaccine composition, comprising:
the recombinant respiratory syncytial virus (RSV) according to claim 1; and
a pharmaceutically acceptable carrier.

15. A bivalent vaccine composition for preventing infection of
respiratory syncytial virus (RSV); and one of measles virus (MV) and Newcastle disease virus (NDV) comprising:
the recombinant respiratory syncytial virus (RSV) according to claim 1; and
a pharmaceutically acceptable carrier.

16. (canceled)
